(19) United States
(12) Patent Application Publication
(10) Pub. No.: US 2020/0325481 A1
(43) Pub. Date: Oct. 15, 2020

(54) ANTISENSE-BASED THERAPEUTICS FOR TARGETING HTRA1 AND METHODS OF USE

(71) Applicant: Gemini Therapeutics Inc., Cambridge, MA (US)

(72) Inventors: Walter Strapps, Cambridge, MA (US); James McLaughlin, Cambridge, MA (US)

(21) Appl. No.: 16/685,136

(22) Filed: Nov. 15, 2019

Related U.S. Application Data
(60) Provisional application No. 62/768,497, filed on Nov. 16, 2018.

(51) Int. Cl. C12N 15/113; A61K 31/713; A61P 27/02

(52) U.S. Cl. CPC C12N 15/1137; A61K 31/713; C12N 2310/3233; C12N 2310/11; C12N 2310/341; A61P 27/02

(57) ABSTRACT

The present disclosure provides compositions and methods for treating, preventing, or inhibiting diseases of the eye. In one aspect, the disclosure provides HTRA1 ASO agents and methods of using the same.

### CROSS-REFERENCE TO RELATED APPLICATIONS

This application claims the benefit of priority from U.S. Provisional Application No. 62/768,497, filed Nov. 16, 2018. The specification of the foregoing application is incorporated herein by reference in its entirety.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: Early AMD; Intermediate AMD; Advanced non-neovascular (“Dry”) AMD; and Advanced neovascular (“Wet”) AMD. Typically, AMD is identified by the focal hyperpigmentation of the retinal pigment epithelium (RPE) and accumulation of drusen deposits. The size and number of drusen deposits typically correlates with AMD severity.

AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040.

There is a need for novel treatments for preventing progression from early to intermediate and/or from intermediate to advanced stages of AMD to prevent loss of vision.

### SUMMARY OF THE DISCLOSURE

In some embodiments, the disclosure provides for an ASO agent that targets an HTRA1 polynucleotide, wherein the HTRA1 polynucleotide encodes an HTRA1 polypeptide or functional fragment thereof. In some embodiments, the HTRA1 polypeptide comprises an amino acid sequence that is at least 80%, 85%, 90%, 93%, 95%, 97%, 98%, 99% or 100% identical to SEQ ID NO: 21. In some embodiments, the ASO agent comprises a nucleotide sequence that is at least 80%, 85%, 90%, 93%, 95%, 97%, 98%, 99% or 100% identical to any one of SEQ ID NOs: 1-20. In some embodiments, the ASO agent comprises the polynucleotide sequence of any of SEQ ID NOs: 1-20, but with 1, 2, 3, 4, 5, 6, 7, 8, 9, or 10 nucleotide modifications as compared to SEQ ID NOs: 1-20. In some embodiments, the ASO agent comprises at least 8, 9, 10, 11, 12, 13, 14, 15, 16, 17, 18 or 19 contiguous nucleotides from a nucleotide sequence that is at least 80%, 85%, 90%, 93%, 95%, 97%, 98%, 99% or 100% identical to any one of SEQ ID NOs: 1-20. In some embodiments, the ASO agent is capable of inhibiting the expression of an HTRA1 polypeptide. In some embodiments, the HTRA1 protein comprises an amino acid sequence that is at least 80%, 85%, 90%, 93%, 95%, 97%, 98%, 99% or 100% identical to SEQ ID NO: 21, or a functional fragment thereof. In some embodiments, the ASO agent is capable of inhibiting the expression of a protein having an amino acid sequence that is at least 80%, 85%, 90%, 93%, 95%, 97%, 98%, 99% or 100% identical to SEQ ID NO: 21, or a functional fragment thereof. In some embodiments, the ASO agent targets an mRNA transcript encoding the HTRA1 protein. In some embodiments, the mRNA transcript encoding the HTRA1 protein comprises a nucleotide sequence that is at least 80%, 85%, 90%, 93%, 95%, 97%, 98%, 99% or 100% identical to the nucleotide sequence of SEQ ID NO: 22 (but wherein thymines are replaced with uracil), or complements thereof. In some embodiments, the ASO agent is capable of inhibiting the expression of HTRA1 protein by at least 5%, 10%, 15%, 25%, 30%, 35%, 40%, 45%, 50%, 55%, 60%, 65%, 70%, 75%, 80%, 85%, 90%, 95% or 100% as compared to the expression level of HTRA1 protein in the absence of the ASO agent. In some embodiments, the ASO agent targets an HTRA1-encoding mRNA transcript. In some embodiments, the ASO agent is capable of reducing HTRA1-encoding mRNA levels in a cell. In some embodiments, the ASO agent is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the ASO agent. In some embodiments, the ASO agent inhibits translation of the HTRA1-encoding mRNA transcript. In some embodiments, the ASO agent targets an HTRA1 pre-mRNA transcript. In some embodiments, the ASO agent is capable of reducing HTRA1 pre-mRNA transcript levels in a cell. In some embodiments, the ASO agent is capable of reducing HTRA1 pre-mRNA transcript levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1 pre-mRNA transcript levels in the same cell type in the absence of the ASO agent. In some embodiments, the ASO agent comprises one or more modified nucleotides. In some embodiments, the one or more modified nucleotides are selected from the group consisting of: deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as NUNA or NUNA), locked nucleotides (represented herein as NLNA or NLNA), 3′-O-methoxy (2′ internucleoside linked) nucleotides (represented herein as 3′-OMen), 2′-F-Arabino nucleotides (represented herein as NfANA or NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e. a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, 2′-alkyl nucleotides, 5-substituted pyrimidines, 6-azapyriinidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine. In some embodiments, one or more nucleotides of the ASO agent are linked by modified internucleoside linkages or backbones. In some embodiments, the modified internucleoside linkage or backbone is selected from the group consisting of: phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, amino alkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, boranophosphates having inverted polarity wherein the adjacent pairs of nucleoside units are linked 3′-5′ to 5′-3′ or 2′-5′ to 5′-2′, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH2 components. In some embodiments, the ASO agent comprises 8 to 30 linked nucleosides and having a nucleobase sequence comprising a complementary region comprising at least 8 contiguous nucleobases complementary to a target region of equal length in an HTRA1 transcript. In some embodiments, the complementary region of the ASO agent is 100% complementary to the target region. In some embodiments, the complementary region of the modified oligonucleotide comprises at least 10 contiguous nucleobases. In some embodiments, the complementary region of the modified oligonucleotide comprises at least 15 contiguous nucleobases. In some embodiments, the complementary region of the modified oligonucleotide comprises at least 20 contiguous nucleobases. In some embodiments, the ASO agent is capable of interfering with polyadenylation of HTRA1 pre-mRNA. In some embodiments, the ASO agent is capable of inhibiting formation of the 5′-cap of HTRA1 pre-mRNA. In some embodiments, the ASO agent is capable of inhibiting splicing of HTRA1 pre-mRNA. In some embodiments, the ASO agent is capable of activating RNase H-dependent degradation of a target HTRA mRNA transcript. In some embodiments, the ASO agent is a gapmer. In some embodiments, the ASO agent is a morpholino.

In some embodiments, the disclosure provides a vector comprising any of the ASO agents disclosed herein. In some embodiments, the vector is a viral vector. In some embodiments, the vector is an AAV vector. In some embodiments, the vector is a non-viral vector. In some embodiments, the vector is a nanoparticle. In some embodiments, the vector is a liposome.

In some embodiments, the disclosure provides a host cell comprising any of the vectors disclosed herein.

In some embodiments, the disclosure provides a method of treating a disease or disorder in a subject in need thereof, wherein the disease or disorder is associated with aberrantly expressed HTRA1, wherein the method comprises administering to the subject any of the ASO agents and/or any of the vectors disclosed herein. In some embodiments, the disclosure provides a method of treating a disease or disorder in a subject in need thereof, wherein HTRA1 is expressed at a level at least 5%, 10%, 25%, 50%, 75%, 100%, 150%, 200%, 250%, 300%, 350%, 400%, 450%, or 500% greater in the subject having the disease or disorder as compared to the level in a control subject not having the disease or disorder, wherein the method comprises administering to the subject any of the ASO agents and/or any of the vectors disclosed herein. In some embodiments, the disclosure provides a method of treating age-related macular degeneration or polypoidal choroidal vasculopathy in a subject in need thereof, wherein the method comprises administering to the subject any of the ASO agents and/or any of the vectors disclosed herein. In some embodiments, the control subject is a subject of the same sex and/or of similar age as the subject having the disease or disorder. In some embodiments, the subject has one or more mutations in the HTRA1 gene. In some embodiments, the one or more mutations are not in the coding sequence for the HTRA1 gene. In some embodiments, the one or more mutations are in 10q26 in a human subject. In some embodiments, the one or more mutations correspond to any one or more of the following polymorphisms in a human subject: rs61871744; rs59616332; rs11200630; rs61871745; rs11200632; rs11200633; rs61871746; rs61871747; rs370974631; rs200227426; rs201396317; rs199637836; rs11200634; rs75431719; rs10490924;

rs144224550; rs36212731; rs36212732; rs36212733; rs3750848; rs3750847; rs3750846; rs566108895; rs3793917; rs3763764; rs11200638; rs1049331; rs2293870; rs2284665; rs60401382; rs11200643; rs58077526; rs932275 and/or rs2142308. In some embodiments, the subject has age-related macular degeneration. In some embodiments, the subject is a human. In some embodiments, the human is at least 40 years of age. In some embodiments, the human is at least 50 years of age. In some embodiments, the human is at least 65 years of age. In some embodiments, the ASO agent is administered locally. In some embodiments, the ASO agent is administered intravitreally. In some embodiments, the ASO agent is administered subretinally. In some embodiments, the ASO agent is administered systemically. In some embodiments, the subject has polypoidal choroidal vasculopathy. In some embodiments, the subject has Wet age-related macular degeneration. In some embodiments, the subject has Dry age-related macular degeneration.

In some embodiments, the disclosure provides for a composition comprising a pharmaceutically acceptable carrier and any of the ASO agents disclosed herein and/or any of the vectors disclosed herein. In some embodiments, the composition is substantially pyrogen free.

### DETAILED DESCRIPTION OF THE DISCLOSURE

The disclosure provides compositions and methods for treating, preventing, or inhibiting diseases of the eye. In one aspect, the disclosure provides for HTRA1 antisense oligomer or “ASO” agents. In another aspect, the disclosure provides methods of treating, preventing, or inhibiting diseases of the eye by intraocularly (e.g., intravitreally) administering an effective amount of any of the ASO agents disclosed herein.

A wide variety of diseases of the eye may be treated or prevented using the ASO agents and methods provided herein. Diseases of the eye that may be treated or prevented using the HTRA1 ASO agents and methods of the disclosure include but are not limited to, glaucoma, macular degeneration (e.g., age-related macular degeneration), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically).

General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the disclosure.

Exemplary methods and materials are described herein, although methods and materials similar or equivalent to those described herein can also be used in the practice or testing of the present disclosure. The materials, methods, and examples are illustrative only and not intended to be limiting.

Definitions

The following terms, unless otherwise indicated, shall be understood to have the following meanings:

As used herein, “antisense oligomer” or “ASO” or “antisense compound” means a compound comprising or consisting of an oligonucleotide at least a portion of which is complementary to a target nucleic acid (e.g., a nucleic acid encoding HTRA1 protein) to which it is capable of hybridizing, resulting in at least one antisense activity.

As used herein, “antisense activity” means any detectable and/or measurable change attributable to the hybridization of an antisense compound to its target nucleic acid. For example, an antisense activity may include activation of RNase H-dependent degradation of a target nucleic acid, inhibition of splicing of a pre-mRNA transcript, inhibition of polyadenylation of a pre-mRNA transcript, and/or inhibition of formation of the 5′-cap of a pre-mRNA transcript.

As used herein, “target transcript”, “target”, or “target nucleic acid” means a nucleic acid molecule to which an antisense compound hybridizes.

As used herein, “mRNA” means an RNA molecule that encodes a protein.

As used herein, “pre-mRNA” means an RNA transcript that has not been fully processed into mRNA. Pre-RNA includes one or more intron.

As used herein, “transcript” means an RNA molecule transcribed from DNA. Transcripts include, but are not limitied to mRNA, pre-mRNA, and partially processed RNA.

As used herein, “HTRA1 transcript” means an RNA molecule transcribed from an HTRA1 gene, and which encodes an HTRA1 protein (e.g., a polypeptide comprising the amino acid sequence of SEQ ID NO: 21). An example of an HTRA1 transcript includes the nucleotide sequence of SEQ ID NO: 22, or a complement thereof, or a nucleotide sequence of SEQ ID NO: 22 in which thymines are replaced with uracil, or a complement thereof.

As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, a “base”, “nucleotide base,” or “nucleobase,” is a heterocyclic pyrimidine or purine compound, which is a standard constituent of all nucleic acids, and includes the bases that form the nucleotides adenine (A), guanine (G), cytosine (C), thymine (T), and uracil (U). A nucleobase may further be modified to include, without limitation, universal bases, hydrophobic bases, promiscuous bases, size-expanded bases, and fluorinated bases. As used herein, the term “nucleotide” can include a modified nucleotide (such as, for example, a nucleotide mimic, abasic residue (Ab), or a surrogate replacement moiety).

As used herein, the terms “sequence” and “nucleotide sequence” mean a succession or order of nucleobases or nucleotides, described with a succession of letters using standard nomenclature.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, and unless otherwise indicated, the term “complementary,” when used to describe a first nucleobase or nucleotide sequence (e.g., ASO agent or targeted mRNA) in relation to a second nucleobase or nucleotide sequence, means the ability of an oligonucleotide or polynucleotide including the first nucleotide sequence to hybridize (form base pair hydrogen bonds under mammalian physiological conditions (or similar conditions in vitro)) and form a duplex or double helical structure under certain standard conditions with an oligonucleotide or polynucleotide including the second nucleotide sequence. Complementary sequences include

Watson-Crick base pairs or non-Watson-Crick base pairs and include natural or modified nucleotides or nucleotide mimics, at least to the extent that the above hybridization requirements are fulfilled. Sequence identity or complementarity is independent of modification.

As used herein, “perfectly complementary” or “fully complementary” means that all (100%) of the nucleobases or nucleotides in a contiguous sequence of a first polynucleotide will hybridize with the same number of nucleobases or nucleotides in a contiguous sequence of a second polynucleotide. The contiguous sequence may comprise all or a part of a first or second nucleotide sequence.

As used herein, “partially complementary” means that in a hybridized pair of nucleobase sequences, at least 70%, but not all, of the bases in a contiguous sequence of a first polynucleotide will hybridize with the same number of bases in a contiguous sequence of a second polynucleotide.

As used herein, “substantially complementary” means that in a hybridized pair of nucleobase sequences, at least 85%, but not all, of the bases in a contiguous sequence of a first polynucleotide will hybridize with the same number of bases in a contiguous sequence of a second polynucleotide. The terms “complementary,” “fully complementary,” “partially complementary,” and “substantially complementary” herein are used with respect to the nucleobase or nucleotide matching between the sense strand and the antisense strand of an ASO agent, or between the antisense strand of an ASO agent and a sequence of a target mRNA (e.g., an HTRA1 mRNA transcript).

As used herein, a “host cell” includes an individual cell or cell culture that can be or has been a recipient for vector(s) for incorporation of polynucleotide inserts. The term host cell may refer to the packaging cell line in which the ASO agent is produced from the plasmid.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein (e.g., HTRA1) or protein subunit translated from the mRNA in a cell, group of cells, tissue, organ, or subject in which the gene is transcribed, is reduced when the cell, group of cells, tissue, organ, or subject is treated with the ASO agents described herein as compared to a second cell, group of cells, tissue, organ, or subject that has not or have not been so treated.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, 45, 50, 55, 60, 65, 70, 75, 80, 85, 90 or 95 years of age.

In one embodiment, the subject has, or is at risk of developing a disease of the eye. A disease of the eye, includes, without limitation, AMD, retinitis pigmentosa, rod-cone dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder. In another embodiment, the subject is in need of optogenetic therapy. In another embodiment, the subject has shown clinical signs of a disease of the eye.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

As used herein, the terms “prevent”, “preventing” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject for an infection, “prevent”, “preventing” and “prevention” refer to the inhibition or a reduction in the development or onset of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of an infection (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of” a substance, a compound or an agent to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In particular embodiments, the cells are RPE cells.

As used herein, the term “capable of” means that the referenced composition (e.g., ASO agent) has the capability to perform a specific function, but that it is not required to be performing that specific function at any specific moment in time. The term “capable of” encompasses instances where the composition is actively performing a specific function.

Each embodiment described herein may be used individually or in combination with any other embodiment described herein.

ASO Agents

HTRA1 is a serine protease that targets a variety of proteins, including extracellular matrix proteins such as fibronectin. Fibronectin fragments resulting from HTRA1 cleavage are able to further induce synovial cells to up-regulate MMPI and MMP3 production. There is evidence that HTRA1 may also degrade proteoglycans, such as aggrecan, decorin and fibromodulin. By cleaving proteoglycans, HTRA1 may release soluble FGF-glycosaminoglycan complexes that promote the range and intensity of FGF signals in the extracellular space. HTRA1 also regulates the availability of insulin-like growth factors (IGFs) by cleaving IGF-binding proteins. Intracellularly, HTRA1 degrades TSC2, leading to the activation of TSC2 downstream targets.

Overexpression of HTRA1 alters the integrity of Bruch's membrane, which permits choroid capillaries to invade across the extracellular matrix in conditions such as wet age-related macular degeneration. Tong et al., 2010, Mol. Vis., 16:1958-81. HTRA1 also inhibits signaling mediated by TGF-beta family members, which may regulate many physiological processes, including retinal angiogenesis and neuronal survival and maturation during development. It has been previously determined that a single-nucleotide polymorphism (rs11200638) in the promoter region of the HTRA1 gene was found to be significantly associated with susceptibility to AMD in various patient populations. Tong et al., 2010.

In certain embodiments, ASO agents of the present disclosure are antisense compounds. Such antisense compounds are capable of hybridizing to a target nucleic acid, resulting in at least one antisense activity. In some embodiments, the antisense activity is activation of RNase H-dependent degradation of a target nucleic acid. In some embodiments, the antisense activity is inhibition of splicing of HTRA1 pre-mRNA. In some embodiments, the antisense activity is interference with polyadenylation of HTRA1 pre-mRNA. In some embodiments, the antisense activity is inhibition of the 5′-cap formation of HTRA1 pre-mRNA.

In certain embodiments, ASO agents specifically hybridize to one or more target nucleic acid. In certain embodiments, a specifically hybridizing antisense compound has a nucleobase sequence comprising a region having sufficient complementarity to a target nucleic acid to allow hybridization and result in antisense activity and insufficient complementarity to any non-target so as to avoid non-specific hybridization to any non-target nucleic acid sequences under conditions in which specific hybridization is desired (e.g., under physiological conditions for in vivo or therapeutic uses, and under conditions in which assays are performed in the case of in vitro assays).

In certain embodiments, the present disclosure provides ASO agents comprising oligonucleotides that are fully complementary to the target nucleic acid (e.g., an HTRA1 transcript) over the entire length of the oligonucleotide. In certain embodiments, oligonucleotides are 99% complementary to the target nucleic acid. In certain embodiments, oligonucleotides are 95% complementary to the target nucleic acid. In certain embodiments, such oligonucleotides are 90% complementary to the target nucleic acid. In certain embodiments, such oligonucleotides are 85% complementary to the target nucleic acid. In certain embodiments, such oligonucleotides are 80% complementary to the target nucleic acid. In certain embodiments, an antisense compound comprises a region that is fully complementary to a target nucleic acid and is at least 80% complementary to the target nucleic acid over the entire length of the oligonucleotide. In certain such embodiments, the region of full complementarity is from 6 to 14 nucleobases in length.

In some embodiments, any of the HTRA1 ASO agents disclosed herein comprise or consist of an oligonucleotide comprising a region that is complementary to a target nucleic acid. In certain embodiments, the target nucleic acid is an endogenous RNA molecule. In some embodiments, the target nucleic acid is an endogenous HTRA1 mRNA transcript. In certain embodiments, the target nucleic acid is a pre-mRNA. In certain embodiments, the target nucleic acid is an HTRA1 transcript. In certain embodiments, the target RNA is an HTRA1 pre-mRNA.

The ASO agents of the present disclosure are antisense molecules that are capable of at least one antisense activity upon hybridization with a target nucleic acid (e.g., an HTRA1 transcript). In some embodiments, any of the ASO agents disclosed herein is capable of interfering with polyadenylation of HTRA1 pre-mRNA. In some embodiments, the ASO agent is capable of inhibiting formation of the 5′-cap of HTRA1 pre-mRNA. In some embodiments, the ASO agent is capable of inhibiting splicing of HTRA1 pre-mRNA. In some embodiments, the ASO agent is capable of activating RNase H-dependent degradation of a target HTRA mRNA transcript.

In some embodiments, any of HTRA1 ASO agents disclosed herein is capable of decreasing proteolytic activity of an HTRA1 protein in a cell, tissue (e.g., eye) or organ. In some embodiments, the HTRA1 ASO agent is capable of decreasing proteolytic activity by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in the absence of the ASO agent. In some embodiments, the ASO agent is capable of reducing the HTRA1 proteolytic activity in a cell by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in the same cell type in the absence of the ASO agent. In some embodiments, the ASO agent is capable of reducing HTRA1 proteolytic activity in an eye by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in an eye in the absence of the ASO agent.

In some embodiments, the ASO agent is capable of reducing HTRA1 cleavage of any one or more HTRA1 substrate. In some embodiments, the HTRA1 substrate is selected from the group consisting of: fibromodulin, clusterin, ADAMS, elastin, vitronectin, a2-macroglobulin, talin-1, fascin, LTBP-1, EFEMP1, and chloride intracellular channel protein. In some embodiments, the ASO agent is capable of reducing HTRA1 cleavage of any one or more regulator of the complement cascade (e.g., vitronectin, fibromodulin or clusterin). In some embodiments, the ASO agent is capable of reducing HTRA1 cleavage of any one or more HTRA1 substrate and/or regulator of the complement cascade by at least 5%, 10%, 15%, 25%, 30%, 35%, 40%, 45%, 50%, 55%, 60%, 65%, 70%, 75%, 80%, 85%, 90%, 95% or 100% as compared to the ability of the HTRA1 to cleave the HTRA1 substrate and/or regulator of the complement cascade in the absence of the ASO agent.

In some embodiments, any of the ASO agents disclosed herein comprise a nucleotide sequence that is at least 80%, 85%, 90%, 93%, 95%, 97%, 98%, 99% or 100% identical to any one of SEQ ID NOs: 1-20. In some embodiments, any of the ASO agents disclosed herein comprise any of the nucleotide sequences disclosed herein, but with at least one or more of any of the nucleotide modifications disclosed herein. In some embodiments, any of the ASO agents disclosed herein comprises the polynucleotide sequence of any of SEQ ID NOs: 1-20, but with 1, 2, 3, 4, 5, 6, 7, 8, 9, or 10 nucleotide modifications as compared to the corresponding SEQ ID NO: 1-20. For example, an ASO agent may comprise the nucleotide sequence of SEQ ID NO: 1, but with 2 nucleotide modifications as compared to SEQ ID NO: 1; or the ASO agent may comprise the nucleotide sequence of SEQ ID NO: 2, but with 1 nucleotide modification as compared to SEQ ID NO: 2. In some embodiments, any of the ASO agents disclosed herein comprises at least 8, 9, 10, 11, 12, 13, 14, 15, 16, 17, 18 or 19 contiguous nucleotides present from a nucleotide sequence that is at least 80%, 85%, 90%, 93%, 95%, 97%, 98%, 99% or 100% identical to any one of SEQ ID NOs: 1-20.

In some embodiments, any of the ASO agents disclosed herein is capable of inhibiting the expression of an HTRA1 protein. In some embodiments, the HTRA1 protein comprises an amino acid sequence that is at least 80%, 85%, 90%, 93%, 95%, 97%, 98%, 99% or 100% identical to SEQ ID NO: 21, or a functional fragment thereof. In some embodiments, any of the ASO agents disclosed herein is capable of inhibiting the expression of a protein having an amino acid sequence that is at least 80%, 85%, 90%, 93%, 95%, 97%, 98%, 99% or 100% identical to SEQ ID NO: 21, or a functional fragment thereof. In some embodiments, any of the ASO agents disclosed herein target an mRNA transcript encoding the HTRA1 protein. In some embodiments, the mRNA transcript encoding the HTRA1 protein comprises a nucleotide sequence that is at least 80%, 85%, 90%, 93%, 95%, 97%, 98%, 99% or 100% identical to the nucleotide sequence of SEQ ID NO: 22 (but wherein thymines are replaced with uracil), or complements thereof. In some embodiments, any of the ASO agents disclosed herein targets an mRNA transcript that is at least 80%, 85%, 90%, 93%, 95%, 97%, 98%, 99% or 100% identical to the nucleotide sequence of SEQ ID NO: 22 (but wherein thymines are replaced with uracil), or complements thereof. In some embodiments, any of the ASO agents disclosed herein is capable of inhibiting the expression of HTRA1 protein by at least 5%, 10%, 15%, 25%, 30%, 35%, 40%, 45%, 50%, 55%, 60%, 65%, 70%, 75%, 80%, 85%, 90%, 95% or 100% as compared to the expression level of HTRA1 protein in the absence of the ASO agent. In some embodiments, the ASO agent is capable of targeting the HTRA1-encoding mRNA for degradation. In some embodiments, the ASO agent is capable of inhibiting splicing of HTRA1 pre-mRNA. In some embodiments, any of the ASO agents disclosed herein is capable of reducing HTRAl-encoding mRNA levels in a cell. In some embodiments, the ASO agent is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the ASO agent.

Modified nucleotides, when used in various polynucleotide or oligonucleotide constructs, can preserve activity of the compound in cells while at the same time increasing the serum stability of these compounds, and can also minimize the possibility of activating interferon activity in humans upon administering of the polynucleotide or oligonucleotide construct.

In some embodiments, an HTRA1 ASO agent is prepared or provided as a salt, mixed salt, or a free-acid. In some embodiments, an HTRA1 ASO agent is prepared as a sodium salt. Such forms are within the scope of the disclosures disclosed herein.

In certain embodiments, ASO agents of the disclosure comprise one or more modified nucleosides comprising a modifed sugar moiety. Such ASO agents comprising one or more sugar-modified nucleosides may have desirable properties, such as enhanced nuclease stability or increased binding affinity with a target nucleic acid relative to ASO agents comprising only nucleosides comprising naturally occurring sugar moieties. In certain embodiments, modified sugar moieties are substitued sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of substituted sugar moieties.

In certain embodiments, modified sugar moieties are substituted sugar moieties comprising one or more substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”)- In certain embodiments, sugar substituents at the 2′ position is selected from allyl, amino, azido, thio, O-allyl, O—C₁-C₁₀ alkyl, O—C₁-C₁₀ substituted alkyl; O—C₁-C₁₀ alkoxy; O—C₁-C₁₀ substituted alkoxy, OCF3, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C₁-C₁₀ alkyl. Examples of sugar substituents at the 5′-position, include, but are not limited to:, 5′-methyl (R or S) 5′-vinyl, and 5′-methoxy.

In certain embodiments, substituted sugars comprise more than one non-bridging sugar substituent, for example, 2′-F-5′-methyl sugar moieties (see, e.g., PCT International Application WO 2008/101157, for additional 5′, 2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, O—C₁-C₁₀ alkoxy; O—C₁-C₁₀ substituted alkoxy, SH, CN, OCN, CF₃, OCF₃, O-alkyl, S-alkyl, N(Rm)-alkyl; O-alkenyl, S-alkenyl, or N(Rm)-alkenyl; O-alkynyl, S-alkynyl, N(Rₘ)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rm)(Rn) or O—CH₂—C(═O)—N(Rₘ)(Rₙ), where each Rm and Rn is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro(NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)3NH₂, CH₂—CH═CH₂, O—CH₂—CH═CH₂, OCH₂CH₂OCH₃, O(CH₂)₂SCH₃, O—(CH₂)₂-O—N(Rm)(Rn), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-substituted nucleoside comprises a sugar moiety comprising a 2′-substituent group selected from F, O—CH₃, and OCH₂CH₂OCH₃.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]n-, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)₃-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845); 4′-C(CH₃)(CH₃)—O-2′ and analogs thereof, (see, e.g., WO2009/006478); 4′-CH₂—N(OCH₃)-2′ and analogs thereof (see, e.g., WO2008/150729); 4′-CH₂-O—N(CH₃)-2′ (see, e.g., US2004/0171570); 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,67); 4′-CH₂—C(H)(CH₃)-2′ (see, e.g., Chattopadhyaya, et al, J. Org. Chem.,2009, 74, 118-134); and 4′-CH₂—C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1 , or 2; n is 1 , 2, 3, or 4; each Rₐ and R_{b} is, independently, H, a protecting group, hydroxyl, C₁-C₁₂ alkyl, substituted C₁-C₁₂ alkyl, C₁-C₁₂ alkenyl, substituted C₁-C₁₂ alkenyl, C₁-C₁₂ alkynyl, substituted C₁-C₁₂ alkynyl, C₅-C₂o aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁ NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) -L-Methyleneoxy (4′-CH₂-O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

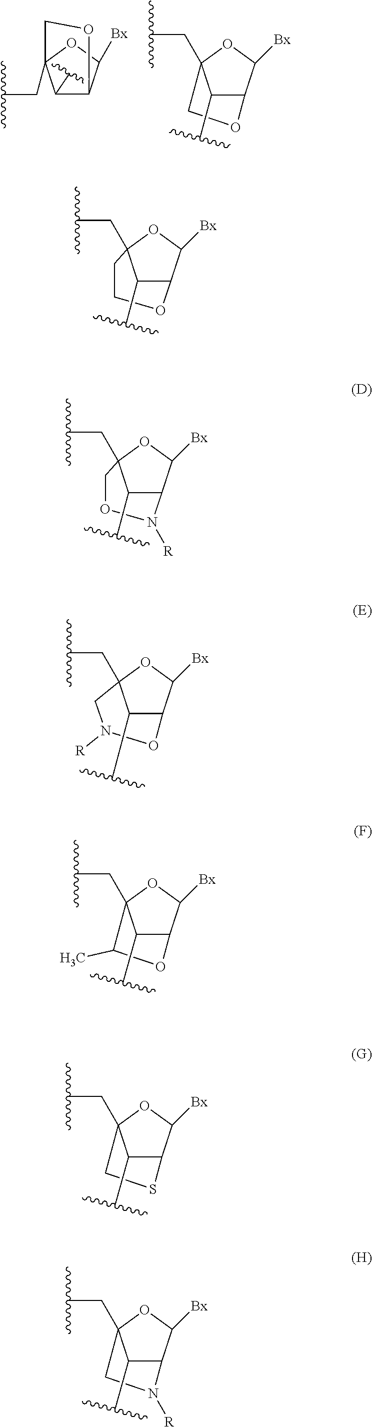

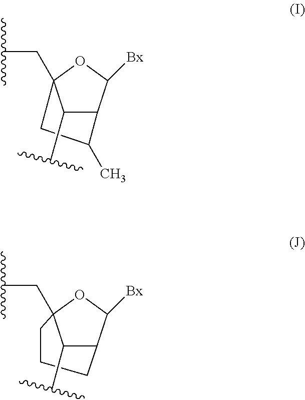

wherein Bx is a nucleobase moiety and R is, independently, H, a protecting group, or C₁C₁₂ alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al. Bioorg. Med. Chem. Lett., 1998, 8, 2219-2222; Singh et al, J. Org. Chem., 1998 63, 10035-10039; Srivastava et al, J. Am. Chem. Soc, 129(26) 8362-8379 (Jul. 4, 2007); Elayadi et al, Curr. Opinion Invens. Drugs, 2001, 2, 558-561 Braasch et al, Chem. Biol., 2001, 8, 1-7; Oram et al, Curr. Opinion Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181 ; U.S. Patent Publication Nos. US2004/0171570 US2007/0287831, and US2008/0039618; U.S. patent Ser. Nos. 12/129,154, 60/989,574, 61/026.995, 61/026,998, 61/056,564, 61/086,231, 61/097,787, and 61/099,844; and per International Applications Nos. PCT/US2008/064591. PCl/US2008/066154, and PCT/US2008/068922.

In certain enibodiments, bicyclic sugar moieties and nucleosides incorporating such bicyclic sugar moieties are further defined by isomeric configuration. For example, a nucleoside comprising a methylene-oxy bridge, may be in the a-L configuration or in the β-D configuration. Previously, a-L-methyleneoxy (4′-CH₂-13 O-2′) bicyclic nucleosides have been incorporated into anti sense oligonucleotides that showed antisense activity (Frieder et al, Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars), 1see, PCI International Application WO 2007/134181 published on Nov. 22, 2007, wherein LNA is substituted with, for example, a 5′-methyl or a 5′-vinyl group).

In certain embodiments, modified sugar moieties are sugar surrogates. In certain such embodiments, the oxygen atom of the naturally occuring sugar is substituted, e.g., with a suffer, carbon or nitrogen atom. In certain such embodiments, such modified sugar moiety also comprises bridging and/or non-bridging substituents as described above. For example, certain sugar surogates comprise a 4′-suffer atom and a substitution at the 2′-position (see, e.g., U.S. Patent Application US2005/0130923) and or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al, J. Org. Chem., 2006, 71, 7731-7740).

In certain embodiments, sugar surrogates comprise rings having other than 5-atoms. For example, in certain embodiments, a sugar surrogate comprises a six-membered tetrahydropyran.

Such tetrahydropyrans may be further modified or substituted, Nucleosides comprising such modified tetrahydropyrans include, but are not limited to, hexitol nucleic acid (HNA), anitol nucleic acid (ANA), manitol nucleic acid (MNA) (see Leumann, C. J. Bioorg. & .Med. Chem. (2002) 10:841-854), fluoro HNA (F-HNA), and those compounds having Formula I

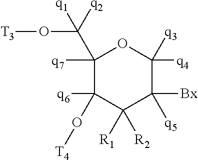

wherein independently for each of said at least one tetrahydropyran nucleoside analog of Formula. I:

Bx is a nucleohase moiety;

T₃ and T₄ are each, independently, an internucleoside linking group linking the tetrahydropyran nucleoside analog to the antisense compound or one of T₃ and T4 is a.n. internucleoside linking group linking the tetrahydropyran nucleoside analog to the antisense compound and the other of T₃ and T₄ is H, a hydroxyl protecting group, a linked conjugate group, or a 5′ or 3′-terminal group, q₁, q₂, q₃, q₄, q₅, q₆, and q₇ are each, independently, H, C₁-C₆ alkyl, substituted C₁-C₆ alkyl, C₂-C₆ alkenyl, substituted C₁-C₆, alkenyl, C₂-C₆ alkynyl, or substituted C₂-C₆ alknyl; and each of R₁ and R₂ is independently selected from among: hydrogen, halogen, substituted or unsubstituted alkoxy, NJ₁J₂, SJ₁, N₃, OC(═X)J, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, the modified THP nucleosides of Formula VII are provided wherein q₁, q₂, q₃, q₄, q₅, q₆, and q₇ are each H. In certain embodiments, at least one of q₁, q₂, q₃, q₄, q₅, q₆, and q₇, is other than H. In certain embodiments, at least one of q₁, q₂, q₃, q₄, q₅, q₆, and q₇ is methyl. In certain embodiments, THP nucleosides of .Formula VII are provided wherein one of R₁ and R₂ is F. In certain embodiments, R₁ is fluoro and R₂ is H, R₁ is methoxy and R₂ is H, and R₁ is methoxyethoxy and R₂ is H. Many other bicyclic and tricyclic sugar and sugar surrogate ring systems are known in the art that can be used to modify nucleosides (see, e.g., review article: Leumann, J. C., Bioorganic & Medicinal Chemistry, 2002, 10, 841-854). In certain embodiments, sugar surrogates comprise rings having more than 5 atoms and more than one heteroatom. For example nucleosides comprising morpholino sugar moieties and their use in ASO agents has been reported (see for example: Braasch et al., Biochemistry, 2002, 41 , 4503-4510; and U.S. Pat. Nos. 5,698,685; 5,166,315; 5,185,444; and 5,034,506). As used here, the term “morpholino” means a sugar having the following structure

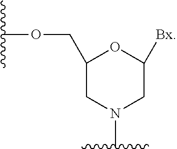

In certain embodiments, inotpholinos may be modified, for example by adding or altering various substituent groups from the above morpholino structure. Such sugar surrogates are refered to herein as “modifed morpholinos.”

Combinations of modifications are also provided without limitation, such as 2′-F-5′-methyl substituted nucleosides (see PCT International Application WO 2008/101157 for other disclosed 5′, 2′-bis substituted nucleosides) and replacement of the ribosyl ring oxygen atom with S and further substitution at the 2′-position (see published U.S. Patent Application US2005-0130923) or alternatively 5′-substitution of a bicyclic nucleic acid (see PCI International Application WO 2007/134181, wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379). Certain Nucleobases in certain embodiments, nucleosides of the present disclosure comprise one or more unmodified nucleobases. In certain embodiments, nucleosides of the present disclosure comprise one or more modifed nucleobases.

In certain embodiments, modified nucleobases are selected from: universal bases, hydrophobic bases, promiscuous bases, size-expanded bases, and fluorinated bases as destined herein. 5-substituted pyrimidines, 6-azapyrimidines and N-2, N-6 and O-6 substituted purines, including 2-aminopropyladenine, 5-propynyluracil; 5-propynyluracil; 5-propynylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded. bases, and fluorinated bases as defined herein. Further modified nucleobases include tricyclic pyrimidines such as phenoxazine cytidine([5,4-b][1,4]henzoxazin-2(3H)-one), phenothiazine cytidine (1H-pyrimido[5,4-b][1,4]benzothiazin-2(3H-one), F-clamps such as a substituted phenoxazine cytidine (e.g. 9-(2-aminoethoxy)-H-pyrirnido[5,4-b][1,4]benzoxazin-2(3M-one), carbazole cytidine (2H-pyrimido[4,5-b]indol-2-one), pyiidoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases nfclude those disclosed in U.S. Pat. No. 3,687,808, those disclosed in The Concise Encyclopedia Of Polymer Science And Engineering, Kroschwitz, Sohn Wiley & Sons, 1990, 858-859; those disclosed by Englisch et al, Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588, 5,830,653 and 6,005,096, each of which is herein incorporated by reference in its entirety.

In certain embodiments, the present disclosure provides ASO agents comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbainate (—O—C(O)(NH)—S—); siloxane (—O—Si(H)₂-O—); and N,N′-dimethylhydrazine (—CH₂—N(CH₃)—N(CH₃)—). Modified linkages, compared to natural phosphodiester linkages, can be used to alter, typically increase, nuclease resistance of the ASO agent. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

In some embodiments, the oligonucleotides described herein contain one or more asymmetric centers and thus give rise to enantiomers, di astereomers, and other stereoisomeric configurations that may be defined, in terms of absolute stereochemistry, as (R) or (S), a or β such as for sugar anomers, or as (D) or (L) such as for amino acids etc. Included in the antisense compounds provided herein are all such possible isomers, as well as their racemic and optically pure forms. Neutral internucleoside linkages include without limitation, phosphotriesters, methylphosphonates, MMI (3′-CH₂—N(CH₃)—O-5′), amide-3 (3′-C(═O)—N(H)-5′), amide-4 (3′-CH₂—N(H)—C(═O)-5′), formacetal (3′-O—CH₂—O-5′), and thioformacetal (3′-S-CH₂—O-5′). Further neutral internucleoside linkages Mdude nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580, Chapters 3 and 4, 40-65). Further neutral internucleoside linkages include nonionic linkages comprising mixed N, O, S and CH₂ component parts.

In some embodiments, an HTRA1 ASO agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as NUNA or NUNA), locked nucleotides (represented herein as NLNA or NLNA), 3′-O-methoxy (2′ internucleoside linked) nucleotides (represented herein as 3′-0Men), 2′-F-Arabino nucleotides (represented herein as NfANA or NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e. a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, and 2′-alkyl nucleotides. It is not necessary for all positions in a given compound to be uniformly modified. Conversely, more than one modification can be incorporated in a single HTRA1 ASO agent or even in a single nucleotide thereof. The ASO can be synthesized and/or modified by methods known in the art. Modification at one nucleotide may be independent of modification at another nucleotide.

Modified nucleobases include synthetic and natural nucleobases, such as 5-substituted pyrimidines, 6-azapyriinidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an ASO agent are modified nucleotides. As used herein, an ASO agent wherein substantially all of the nucleotides present are modified nucleotides is an ASO agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides. In some embodiments, one or more nucleotides of an ASO agent is a ribonucleotide.

In some embodiments, one or more nucleotides of an HTRA1 ASO agent are linked by non-standard linkages or backbones (i.e., modified internucleoside linkages or modified backbones). Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, amino alkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an HTRA1 ASO agent can contain 1, 2, 3, 4, 5, or 6 phosphorothioate linkages, an antisense strand of an HTRA1 ASO agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an HTRA1 ASO agent can contain 1, 2, 3, or 4 phosphorothioate linkages, an antisense strand of an HTRA1 ASO agent can contain 1, 2, 3, or 4 phosphorothioate linkages, or both the sense strand and the antisense strand independently can contain 1, 2, 3, or 4 phosphorothioate linkages.

In some embodiments, an HTRA1 ASO agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, an HTRA1 ASO agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, an HTRA1 ASO agent contains at least two phosphorothioate internucleoside linkages in the sense strand and three or four phosphorothioate internucleoside linkages in the antisense strand.

In some embodiments, any of the ASO agents disclosed herein contains one or more modified nucleotides and one or more modified internucleoside linkages. In some embodiments, a 2′-modified nucleoside is combined with modified internucleoside linkage.

In some embodiments, any of the ASO agents disclosed herein (e.g., an HTRA1 ASO agent) is conjugated to one or more non-nucleotide groups including, but not limited to, a targeting group, linking group, delivery polymer, or a delivery vehicle. In some embodiments, the non-nucleotide group can enhance targeting, delivery or attachment of the ASO agent. In some embodiments, the non-nucleotide group can be covalently linked to the 3′ and/or 5′ end of either the sense strand and/or the antisense strand. In some embodiments, an HTRA1 ASO agent contains a non-nucleotide group linked to the 3′ and/or 5′ end of the sense strand. In some embodiments, a non-nucleotide group is linked to the 5′ end of an HTRA1 ASO agent sense strand. In some embodiments, a non-nucleotide group can be linked directly or indirectly to the ASO agent via a linker/linking group. In some embodiments, a non-nucleotide group is linked to the ASO agent via a labile, cleavable, or reversible bond or linker. In some embodiments, a non-nucleotide group enhances the pharmacokinetic or biodistribution properties of an ASO agent or conjugate to which it is attached to improve cell- or tissue-specific distribution and cell-specific uptake of the ASO agent or conjugate. In some embodiments, a non-nucleotide group enhances endocytosis of the ASO agent.

In some embodiments, a targeting group or targeting moiety can enhance the pharmacokinetic or biodistribution properties of a conjugate or ASO agent to which they are attached to improve cell-specific distribution and cell-specific uptake of the conjugate or ASO agent. In some embodiments, a targeting group can be monovalent, divalent, trivalent, tetravalent, or have higher valency for the target to which it is directed. In some embodiments, representative targeting groups include, without limitation, compounds with affinity to cell surface molecules, cell receptor ligands, haptens, antibodies, monoclonal antibodies, antibody fragments, and antibody mimics with affinity to cell surface molecules. In some embodiments, a targeting group is linked to an ASO agent using a linker, such as a PEG linker or one, two, or three abasic and/or ribitol (abasic ribose) residues, which in some instances can serve as linkers. In some embodiments, a targeting group comprises a galactose derivative cluster.

In some embodiments, any of the HTRA1 ASO agents described herein can be synthesized having a reactive group, such as an amine group, at the 5′-terminus. In some embodiments, the reactive group can be used to subsequently attach a targeting group using methods typical in the art.

In some embodiments, a linking group is conjugated to any of the ASO agents disclosed herein.

In some embodiments, the linking group facilitates covalent linkage of the agent to a targeting group or delivery polymer or delivery vehicle. In some embodiments, the linking group can be linked to the 3′ or the 5′ end of the ASO agent sense strand or antisense strand. In some embodiments, the linking group is linked to the ASO agent sense strand. In some embodiments, the linking group is conjugated to the 5′ or 3′ end of an ASO agent sense strand. In some embodiments, a linking group is conjugated to the 5′ end of an ASO agent sense strand. Examples of linking groups, can include, but are not limited to: reactive groups such a primary amines and alkynes, alkyl groups, abasic nucleotides, ribitol (abasic ribose), and/or PEG groups.

In some embodiments, a linker or linking group is a connection between two atoms that links one chemical group (such as an ASO agent) or segment of interest to another chemical group (such as a targeting group or delivery polymer) or segment of interest via one or more covalent bonds. A labile linkage contains a labile bond. A linkage may optionally include a spacer that increases the distance between the two joined atoms. A spacer can further add flexibility and/or length to the linkage. Spacers can include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity. Suitable “moderately stringent conditions” include prewashing in a solution of 5× SSC, 0.5% SDS, 1.0 mM EDTA (pH 8.0); hybridizing at 50° C-65° C., 5× SSC, overnight; followed by washing twice at 65° C. for 20 minutes with each of 2×, 0.5× and 0.2× SSC containing 0.1% SDS. As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Fico11/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 0.075 M sodium citrate), 50 mM sodium phosphate (pH 6.8), 0.1% sodium pyrophosphate, 5 x Denhardt's solution, sonicated salmon sperm DNA (50 μg/ml), 0.1% SDS, and 10% dextran sulfate at 42° C., with washes at 42° C. in 0.2× SSC (sodium chloride/sodium citrate) and 50% formamide at 55° C., followed by a high-stringency wash consisting of 0.1× SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The nucleic acids/polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence. In other embodiments, nucleic acids of the disclosure also include nucleotide sequences that hybridize under highly stringent conditions to the nucleotide sequences set forth in any of the sequences of SEQ ID NOs: 1-20 or 22, or sequences complementary thereto. One of ordinary skill in the art will readily understand that appropriate stringency conditions which promote DNA hybridization can be varied. For example, one could perform the hybridization at 6.0× sodium chloride/sodium citrate (SSC) at about 45° C., followed by a wash of 2.0× SSC at 50° C. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6 x SSC at room temperature followed by a wash at 2× SSC at room temperature.

Isolated nucleic acids which differ due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. One skilled in the art will appreciate that these variations in one or more nucleotides (up to about 3-5% of the nucleotides) of the nucleic acids encoding a particular protein may exist among members of a given species due to natural allelic variation. Any and all such nucleotide variations and resulting amino acid polymorphisms are within the scope of this disclosure.

In certain embodiments, the present disclosure provides ASO agents comprising oligonucleotides. In certain embodiments, such oligonucleotides comprise one or more chemical modification. In certain embodiments, chemically modified oligonucleotides comprise one or more modified nucleosides. In certain embodiments, chemically modified oligonucleotides comprise one or more modified nucleosides comprising modified sugars. In certain embodiments, chemically modified oligonucleotides comprise one or more modified nucleosides comprising one or more modified nucleobases. In certain embodiments, chemically modified oligonucleotides comprise one or more modified internucleoside linkages. In certain embodiments, the chemically modifications (sugar modifications, nucleobase modifications, and/or linkage modifications) define a pattern or motif. In certain embodiments, the patterns of chemical modifications of sugar moieties, internucleoside linkages, and nucleobases are each independent of one another. Thus, an oligonucleotide may be described by its sugar modification motif, internucleoside linkage motif and/or nucleobase modification motif (as used herein, nucleobase modification motif describes the chemical modifications to the nucleobases independent of the sequence of nucleobases).

In certain embodiments, oligonucleotides comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having a gapmer sugar modification motif, which comprises two external regions or “wings” and an internal region or “gap.” The three regions of a gapmer motif (the 5 ′-wing, the gap, and the 3 ′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar modification motifs of the two wings are the same as one another (symmetric gapmer). In certain embodiments, the sugar modification motifs of the 5′-wing differs from the sugar modification motif of the 3′-wing (asymmetric gapmer). In certain embodiments, oligonucleotides comprise 2′-MOE modified nucleosides in the wings and 2′-F modified nucleosides in the gap.

In certain embodiments, oligonucleotides are fully modified. In certain such embodiments, oligonucleotides are uniformly modified. In certain embodiments, oligonucleotides are uniform 2′-MOE. In certain embodiments, oligonucleotides are uniform 2′-F. In certain embodiments, oligonucleotides are uniform morpholino. In certain embodiments, oligonucleotides are uniform BNA. In certain embodiments, oligonucleotides are uniform LNA. In certain embodiments, oligonucleotides are uniform cEt.

In certain embodiments, oligonucleotides comprise a uniformly modified region and additional nucleosides that are unmodified or differently modified. In certain embodiments, the uniformly modified region is at least 5, 10, 15, or 20 nucleosides in length. In certain embodiments, the uniform region is a 2′-MOE region. In certain embodiments, the uniform region is a 2′-F region. In certain embodiments, the uniform region is a morpholino region. In certain embodiments, the uniform region is a BNA region. In certain embodiments, the uniform region is a LNA region. In certain embodiments, the uniform region is a cEt region.

In certain embodiments, the oligonucleotide does not comprise more than 4 contiguous unmodified 2′-deoxynucleosides. In certain circumstances, antisesense oligonucleotides comprising more than 4 contiguous 2′-deoxynucleosides activate RNase H, resulting in cleavage of the target RNA. In certain embodiments, such cleavage is avoided by not having more than 4 contiguous 2′-deoxynucleosides, for example, where alteration of splicing and not cleavage of a target RNA is desired.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif, as described above for sugar modification motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The sugar modification motif is independently selected, so such oligonucleotides having a gapped internucleoside linkage motif may or may not have a gapped sugar modification motif and if it does have a gapped sugar motif, the wing and gap lengths may or may not be the same.

In certain embodiments, oligonucleotides comprise a region having an alternating internucleoside linkage motif. In certain embodiments, oligonucleotides of the present disclosure comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate interaucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise chemical modifications to nucleobases arranged along the oligonucleotide or region thereof in a defined pattern or nucleobases modification motif. In certain such embodiments, nucleobase modifications are arranged in a gapped motif. In certain embodiments, nucleobase modifications are arranged in an alternating motif. In certain embodiments, each nucleobase is modified. In certain embodiments, none of the nucleobases is chemically modified.

In certain embodiments, oligonucleotides comprise a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5 ′-end of the oligonucleotide. In certain embodiments the block is within 3 nucleotides of the 5′-end of the oligonucleotide.

In certain embodiments, nucleobase modifications are a function of the natural base at a particular position of an oligonucleotide. For example, in certain embodiments each purine or each pyrimidine in an oligonucleotide is modified. In certain embodiments, each adenine is modified. In certain embodiments, each guanine is modified. In certain embodiments, each thymine is modified. In certain embodiments, each cytosine is modified. In certain embodiments, each uracil is modified.

In certain embodiments, some, all, or none of the cytosine moieties in an oligonucleotide are 5-methyl cytosine moieties. Herein, 5-methyl cytosine is not a “modified nucleobase.” Accordingly, unless otherwise indicated, unmodified nucleobases include both cytosine residues having a 5-methyl and those lacking a 5 methyl. In certain embodiments, the methylation state of all or some cytosine nucleobases is specified.

In certain embodiments, the present disclosure provides ASO agents including oligonucleotides of any of a variety of ranges of lengths. In certain embodiments, the disclosure provides ASO agents or oligonucleotides consisting of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, the disclosure provides ASO agents which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an ASO agent or oligonucleotide is limited, whether to a range or to a specific number, the ASO agent or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents. In certain embodiments, a gapmer oligonucleotide has any of the above lengths.

One of skill in the art will appreciate that certain lengths may not be possible for certain motifs.

For example: a gapmer having a 5′-wing region consisting of four nucleotides, a gap consisting of at least six nucleotides, and a 3′-wing region consisting of three nucleotides cannot have an overall length less than 13 nucleotides. Thus, one would understand that the lower length limit is 13 and that the limit of 10 in “10-20” has no effect in that embodiment. Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range. For example, an oligonucleotide consisting of 20-25 linked nucleosides comprising a 5′-wing consisting of 5 linked nucleosides; a 3′-wing consisting of 5 linked nucleosides and a central gap consisting of 10 linked nucleosides (5+5+10=20) may have up to 5 nucleosides that are not part of the 5′-wing, the 3′-wing, or the gap (before reaching the overall length limitation of 25). Such additional nucleosides may be 5′ of the 5′-wing and/or 3′ of the 3′ wing.

In certain embodiments, oligonucleotides of the present disclosure are characterized by their sugar motif, internucleoside linkage motif, nucleobase modification motif and overall length. In certain embodiments, such parameters are each independent of one another. Thus, each internucleoside linkage of an oligonucleotide having a gapmer sugar motif may be modified or unmodified and may or may not follow the gapmer modification pattern of the sugar modifications. Thus, the internucleoside linkages within the wing regions of a sugar-gapmer may be the same or different from one another and may be the same or different from the internucleoside linkages of the gap region. Likewise, such sugar-gapmer oligonucleotides may comprise one or more modified nucleobase independent of the gapmer pattern of the sugar modifications. Herein if a description of an oligonucleotide or ASO agent is silent with respect to one or more parameter, such parameter is not limited. Thus, an ASO agent described only as having a gapmer sugar motif without further description may have any length, internucleoside linkage motif, and nucleobase modification motif. Unless otherwise indicated, all chemical modifications are independent of nucleobase sequence.

In certain embodiments, ASO agents are modified by attachment of one or more conjugate groups. In general, conjugate groups modify one or more properties of the attached ASO agent including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an ASO agent, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 11 11-11 18; Kabanov et al, FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651 -3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651 -3654), a palmityl moiety (Mishra et al., Biochim. Biophys. Acta, 1995, 1264, 229-237), or an octadecylamine or hexylamino-carbonyl-oxycholesterol moiety (Crooke et al, J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In certain embodiments, conjugate groups are directly attached to oligonucleotides in ASO agents. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an ASO agent. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some non-limiting examples of conjugate linking moieties include pyrrolidine, 8-amino-3,6-dioxaoctanoic acid (ADO), succinimidyl 4-(N-maleimidomethyl) cyclohexane-l-carboxylate (SMCC) and 6-aminohexanoic acid (AHEX or AHA). Other linking groups include, but are not limited to, substituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an ASO agent. In certain embodiments, conjugate groups are near the 3′-end. In certain embodiments, conjugates are attached at the 3′-end of an ASO agent, but before one or more terminal group nucleosides. In certain embodiments, conjugate groups are placed within a terminal group.

In certain embodiments, the present disclosure provides ASO agents. In certain embodiments, ASO agents comprise an oligonucleotide. In certain embodiments, an ASO agent comprises an oligonucleotide and one or more conjugate and/or terminal groups. Such conjugate and/or terminal groups may be added to oligonucleotides having any of the chemical motifs discussed above. Thus, for example, an ASO agent comprising an oligonucleotide having region of alternating nucleosides may comprise a terminal group.

Pharmaceutical Compositions

Also provided herein are pharmaceutical compositions comprising an ASO agent, and a pharmaceutically acceptable carrier. The pharmaceutical compositions may be suitable for any mode of administration described herein; for example, by intravitreal administration.

In some embodiments, use of any of the ASO agents disclosed herein for treating retinal diseases, such as LCA, retinitis pigmentosa, and age-related macular degeneration require the localized delivery of the ASO agent to the cells in the retina. In some embodiments, the cells that will be the treatment target in these diseases are either the photoreceptor cells in the retina or the cells of the RPE underlying the neurosensory retina.

In some embodiments, the pharmaceutical compositions comprising any of the ASO agents described herein and a pharmaceutically acceptable carrier are suitable for administration to a human subject. Such carriers are well known in the art (see, e.g., Remington's Pharmaceutical Sciences, 15th Edition, pp. 1035-1038 and 1570-1580). In some embodiments, the pharmaceutical compositions comprising any of the ASO agents described herein and a pharmaceutically acceptable carrier is suitable for ocular injection. In some embodiments, the pharmaceutical composition is suitable for intravitreal injection. In some embodiments, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, any of the ASO agents disclosed herein is formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Patent Publication No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid).

In certain embodiments of the methods described herein, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Routes of administration may be combined, if desired.

In some embodiments, any of the ASO agents/pharmaceutical compositions disclosed herein are administered to a patient such that they target cells of any one or more layers or regions of the retina or macula. For example, the compositions disclosed herein target cells of any one or more layers of the retina, including the inner limiting membrane, the nerve fiber layer, the ganglion cell layer (GCL), the inner plexiform layer, the inner nuclear layer, the outer plexiform layer, the outer nuclear layer, the external limiting membrane, the layer of rods and cones, or the retinal pigment epithelium (RPE). In some embodiments, the compositions disclosed herein target glial cells of the GCL, Muller cells, and/or retinal pigment epithelial cells. In some embodiments, the compositions disclosed herein targets cells of any one or more regions of the macula including, for example, the umbo, the foveolar, the foveal avascular zone, the fovea, the parafovea, or the perifovea. In some embodiments, the route of administration does not specifically target neurons. In some embodiments, the route of administration is chosen such that it reduces the risk of retinal detachment in the patient (e.g., intravitreal rather than subretinal administration). In some embodiments, intravitreal administration is chosen if the ASO agent/composition is to be administered to an elderly adult (e.g., at least 60 years of age). In particular embodiments, any of the ASO agents/pharmaceutical compositions disclosed herein are administered to a subject intravitreally. Procedures for intravitreal injection are known in the art (see, e.g., Peyman, G. A., et al. (2009) Retina 29(7):875-912 and Fagan, X.J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antiobiotic. The eye may also be rinsed to remove excess sterilizing agent.

Furthermore, in certain embodiments it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

The composition may be delivered in a volume of from about 0.1 μL to about 1 mL, including all numbers within the range, depending on the size of the area to be treated, the viral titer used (if the ASO agent is administered using a viral vector), the route of administration, and the desired effect of the method. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL.

In some embodiments, any of the ASO agents disclosed herein is administered via a vector. In some embodiments, the vector is a viral vector. In some embodiments, the viral vector is a retrovirus, lentivirus, or baculovirus vector. In some embodiments, the viral vector is an adenoviral vector. In particular embodiments, the viral vector is an AAV vector. A variety of rAAV vectors may be used to deliver the desired ASO agent to the eye and to direct its expression. More than 30 naturally occurring serotypes of AAV from humans and non-human primates are known. Many natural variants of the AAV capsid exist, and an rAAV vector of the disclosure may be designed based on an AAV with properties specifically suited for ocular cells.

Recombinant AAV vectors of the present disclosure may be generated from a variety of adeno-associated viruses. For example, ITRs from any AAV serotype are expected to have similar structures and functions with regard to replication, integration, excision and transcriptional mechanisms. Examples of AAV serotypes include AAV1, AAV2, AAV3, AAV4, AAV5, AAV6, AAV7, AAV8, AAV9, AAV10, AAV11 and AAV12. In some embodiments, the rAAV vector is generated from serotype AAV1, AAV2, AAV4, AAV5, or AAV8. These serotypes are known to target photoreceptor cells or the retinal pigment epithelium. In particular embodiments, the rAAV vector is generated from serotype AAV2. In certain embodiments, the AAV serotypes include AAVrh8, AAVrh8R or AAVrh10. It will also be understood that the rAAV vectors may be chimeras of two or more serotypes selected from serotypes AAV1 through AAV12. The tropism of the vector may be altered by packaging the recombinant genome of one serotype into capsids derived from another AAV serotype. In some embodiments, the ITRs of the rAAV virus may be based on the ITRs of any one of AAV1-12 and may be combined with an AAV capsid selected from any one of AAV1-12, AAV-DJ, AAV-DJ8, AAV-DJ9 or other modified serotypes. In certain embodiments, any AAV capsid serotype may be used with the vectors of the disclosure. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In certain embodiments, the AAV capsid serotype is AAV2.

Desirable AAV fragments for assembly into vectors may include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments maybe used, alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the disclosure. In some embodiments, the AAV is AAV2/5. In another embodiment, the AAV is AAV2/8. When pseudotyping an AAV vector, the sequences encoding each of the essential rep proteins may be supplied by different AAV sources (e.g., AAV1, AAV2, AAV3, AAV4, AAV5, AAV6, AAV7, AAV8). For example, the rep78/68 sequences may be from AAV2, whereas the rep52/40 sequences may be from AAV8.

In one embodiment, the vectors of the disclosure contain, at a minimum, sequences encoding a selected AAV serotype capsid, e.g., an AAV2 capsid or a fragment thereof. In another embodiment, the vectors of the disclosure contain, at a minimum, sequences encoding a selected AAV serotype rep protein, e.g., AAV2 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV2 origin. In certain embodiments, the vectors may comprise rep sequences from an AAV serotype which differs from that which is providing the cap sequences. In some embodiments, the rep and cap sequences are expressed from separate sources (e.g., separate vectors, or a host cell and a vector). In some embodiments, these rep sequences are fused in frame to cap sequences of a different AAV serotype to form a chimeric AAV vector, such as AAV2/8 described in US Patent No. 7,282,199, which is incorporated by reference herein. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In some embodiments, the cap is derived from AAV2.

In some embodiments, any of the vectors disclosed herein includes a spacer, i.e., a DNA sequence interposed between the promoter and the rep gene ATG start site. In some embodiments, the spacer may be a random sequence of nucleotides, or alternatively, it may encode a gene product, such as a marker gene. In some embodiments, the spacer may contain genes which typically incorporate start/stop and polyA sites. In some embodiments, the spacer may be a non-coding DNA sequence from a prokaryote or eukaryote, a repetitive non-coding sequence, a coding sequence without transcriptional controls or a coding sequence with transcriptional controls. In some embodiments, the spacer is a phage ladder sequences or a yeast ladder sequence. In some embodiments, the spacer is of a size sufficient to reduce expression of the rep78 and rep68 gene products, leaving the rep52, rep40 and cap gene products expressed at normal levels. In some embodiments, the length of the spacer may therefore range from about 10 bp to about 10.0 kbp, preferably in the range of about 100 bp to about 8.0 kbp. In some embodiments, the spacer is less than 2 kbp in length.

In certain embodiments, the capsid is modified to improve therapy. The capsid may be modified using conventional molecular biology techniques. In certain embodiments, the capsid is modified for minimized immunogenicity, better stability and particle lifetime, efficient degradation, and/or accurate delivery of the ASO agent. In some embodiments, the modification or mutation is an amino acid deletion, insertion, substitution, or any combination thereof in a capsid protein. A modified polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features. A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), Ile (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gin; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a I3-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile;(2) Polar without charge: Cys, Ser, Thr, Asn, Gln;(3) Acidic (negatively charged): Asp, Glu;(4) Basic (positively charged): Lys, Arg;(5) Residues that influence chain orientation: Gly, Pro; and(6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline). In some embodiments, one or more amino acid substitutions are introduced into one or more of VP1, VP2 and VP3. In one aspect, a modified capsid protein comprises 1, 2, 3, 4, 5, 6, 7, 8, 9, 10, 11, 12, 13, 14, or 15 conservative or non-conservative substitutions relative to the wild-type polypeptide. In another aspect, the modified capsid polypeptide of the disclosure comprises modified sequences, wherein such modifications can include both conservative and non-conservative substitutions, deletions, and/or additions, and typically include peptides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

In some embodiments, the recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, any of the ASO agents disclosed herein is administered to a cell, tissue, organ, or subject using a non-viral vector. In some embodiments, the non-viral vector is a cationic lipid- and/or polymer-based system. In some embodiments, the non-viral vector is a liposome or a nanoparticle. In some embodiments, any of the ASO agents is administered to a cell, tissue, organ or subject by means of: sheddable ternary nanoparticles, lyophilized nanosome formulations, multicomponent synthetic polymers with viral-mimetic chemistry, scFv-mediated delivery, targeted polymeric micelles, biscarbamate cross-linked low molecular weight PEI, carbonate apatite-mediated delivery, dendronized gold nanoparticles, glutathione-responsive nano-transporter, gelatin nanospheres, cationic lipbenzamides, lipid modified triblock PAMAM-based nanocarriers, PAMAM dendrimer conjugates with cyclodextrins, GC-PEI nanoparticles, hemifluorinated polycationic lipids, PEI-based vector systems, glycopolymer-stabilized gold nanoparticles, nanogels and PCI, BBN-oligonucleotide conjugate, chlorotoxin bound magnetic nanovector, oral protein therapy, pH-sensitive carbonate apatite, amphoteric agmatine containing polyamidoamines, biodegradable dextran nanogels, dendrimer, poly(amine-co-esters), multilayered coated gold nanoparticles, biodegradable amphiphilic and cationic triblock copolymer, carbonate apatite nano-composites, polymeric vector incorporating endosomolytic oligomeric sulfonamide, lipid derivatives carrying amino and triazolyl groups, functional lipopolyamine, GPI modification, CADY self-assembling peptide-based nanoparticles, fluorescent PAMAM dendrimer, an injectable scaffold, amino-ethoxilated fluorinated amphiphile, and tyrosine trimers stabilized pDNA and polyplexes.

Methods of Treatment/Prophylaxis

Described herein are various methods of preventing, treating, arresting progression of or ameliorating the ocular disorders and retinal changes associated therewith. Generally, the methods include administering to a mammalian subject in need thereof, an effective amount of a composition comprising any of the ASO agents disclosed herein. Any of the ASO agents disclosed herein are useful in the methods described below.

In some embodiments, any of the ASO agents disclosed herein are for use in treating retinal diseases, such as LCA, retinitis pigmentosa, and age-related macular degeneration may require the localized delivery of the ASO agent to the cells in the retina. In some embodiments, the cells that will be the treatment target in these diseases are either the photoreceptor cells in the retina or the cells of the RPE underlying the neurosensory retina. In some embodiments, delivering any of the ASO agents disclosed herein to these cells requires injection into the subretinal space between the retina and the RPE. In some embodiments, any of the ASO agents disclosed herein are administered intravitreally or intravenously.

In a certain aspect, the disclosure provides a method of treating a subject having age-related macular degeneration (AMD), comprising the step of administering to the subject any of the ASO agents of the disclosure. In some embodiments, the AMD is any one of Early AMD; Intermediate AMD; Advanced non-neovascular (“Dry”) AMD; or Advanced neovascular

(“Wet”) AMD. In some embodiments, the disclosure provides for methods of treating a subject with Wet AMD. In some embodiments, the disclosure provides for methods of treating a subject with Dry AMD. In some embodiments, the disclosure provides for methods of treating a subject with polyploidal choroidal vasculopathy (PCV). In some embodiments, the subject has geographic atrophy.

In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic.

In some embodiments, any of HTRA1 ASO agents disclosed results in a decrease of proteolytic activity of an HTRA1 protein in a cell, tissue, organ (e.g., eye) or subject. In some embodiments, the HTRA1 ASO agent are capable of decreasing proteolytic activity by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in the absence of the ASO agent. In some embodiments, the ASO agent is capable of decreasing HTRA1 proteolytic activity in a cell by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in the same cell type in the absence of the ASO agent. In some embodiments, the ASO agent is capable of reducing HTRA1 proteolytic activity in an eye by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in an eye in the absence of the ASO agent.

In some embodiments, the ASO agent is capable of reducing HTRA1 cleavage of any one or more HTRA1 substrate. In some embodiments, the HTRA1 substrate is selected from the group consisting of: fibromodulin, clusterin, ADAMS, elastin, vitronectin, α2-macroglobulin, talin-1, fascin, LTBP-1, EFEMPL and chloride intracellular channel protein. In some embodiments, the ASO agent is capable of reducing HTRA1 cleavage of any one or more regulator of the complement cascade (e.g., vitronectin, fibromodulin or clusterin). In some embodiments, the ASO agent is capable of reducing HTRA1 cleavage of any one or more HTRA1 substrate and/or regulator of the complement cascade by at least 5%, 10%, 15%, 25%, 30%, 35%, 40%, 45%, 50%, 55%, 60%, 65%, 70%, 75%, 80%, 85%, 90%, 95% or 100% as compared to the ability of the HTRA1 to cleave the HTRA1 substrate and/or regulator of the complement cascade in the absence of the ASO agent.

In some embodiments, any of the ASO agents disclosed herein is capable of inhibiting the expression of an HTRA1 protein in a cell, tissue, organ (e.g., eye) or subject. In some embodiments, the HTRA1 protein comprises an amino acid sequence that is at least 80%, 85%, 90%, 93%, 95%, 97%, 98%, 99% or 100% identical to SEQ ID NO: 21, or a functional fragment thereof. In some embodiments, any of the ASO agents disclosed herein is capable of inhibiting the expression of a protein having an amino acid sequence that is at least 80%, 85%, 90%, 93%, 95%, 97%, 98%, 99% or 100% identical to SEQ ID NO: 21, or a functional fragment thereof. In some embodiments, any of the ASO agents disclosed herein is capable of targeting an mRNA transcript encoding the HTRA1 protein. In some embodiments, the mRNA transcript encoding the HTRA1 protein comprises a nucleotide sequence that is at least 80%, 85%, 90%, 93%, 95%, 97%, 98%, 99% or 100% identical to the nucleotide sequence of SEQ ID NO: 22 (but wherein thymines are replaced with uracil), or complements thereof. In some embodiments, any of the ASO agents disclosed herein is capable of targeting an mRNA transcript that is at least 80%, 85%, 90%, 93%, 95%, 97%, 98%, 99% or 100% identical to the nucleotide sequence of SEQ ID NO: 22 (but wherein thymines are replaced with uracil), or complements thereof. In some embodiments, any of the ASO agents disclosed herein is capable of inhibiting the expression of HTRA1 protein by at least 5%, 10%, 15%, 25%, 30%, 35%, 40%, 45%, 50%, 55%, 60%, 65%, 70%, 75%, 80%, 85%, 90%, 95% or 100% as compared to the expression level of HTRA1 protein in the absence of the ASO agent. In some embodiments, any of the ASO agents disclosed herein is capable of reducing HTRAl-encoding mRNA levels in a cell. In some embodiments, the ASO agent is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the ASO agent.

In some embodiments, any of the ASO agents disclosed herein is administered to cell(s) or tissue(s) in a test subject. In some embodiments, the cell(s) or tissue(s) in the test subject express a higher level of HTRA1 than expressed in the same cell type or tissue type in a reference control subject or population of reference control subjects. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with activation of the complement cascade. In some embodiments, the reference control subject does not have macular degeneration. In some embodiments, the eye or a specific cell type of the eye (e.g., cells in the foveal region) in the test subject express at least 500%, 400%, 300%, 250%, 200%, 150%, 100%, 95%, 90%, 80%, 70%, 60%, 50%, 40%, 30%, 20%, 10%, 5%, or 1% more HTRA1 as compared to the levels in the reference control subject or population of reference control subjects. In some embodiments, the eye or a specific cell type of the eye (e.g., cells in the foveal region) in the test subject express an HTRA1 gene having any of the mutations disclosed herein. In some embodiments, the eye or a specific cell type of the eye (e.g., cells in the foveal region) in the reference control subject do not express a HTRA1 gene having any of the HTRA1 mutations disclosed herein. In some embodiments, administration of any of the ASO agents disclosed herein in the cell(s) or tissue(s) of the test subject results in a decrease in levels of HTRA1 protein or functional HTRA1 protein. In some embodiments, administration of any of the ASO agents disclosed herein in the cell(s) or tissue(s) of the test subject results in a decrease in levels of HTRA1 protein or functional HTRA1 protein such that the decreased levels are within 90%, 80%, 70%, 60%, 50%, 40%, 30%, 20%, 10%, 5%, or 1% of, or are the same as, the levels of HTRA1 protein or functional HTRA1 protein expressed by the same cell type or tissue type in the reference control subject or population of reference control subjects. In some embodiments, administration of any of the ASO agents disclosed herein in the cell(s) or tissue(s) of the test subject results in a decrease in levels of HTRA1 protein or functional HTRA1 protein, but the decreased levels of HTRA1 protein or functional HTRA1 protein are not below the levels of HTRA1 protein or functional HTRA1 protein expressed by the same cell type or tissue type in the reference control subject or population of reference control subjects. In some embodiments, administration of any of the ASO agents disclosed herein in the cell(s) or tissue(s) of the test subject results in a decrease in levels of HTRA1 protein or functional HTRA1 protein, but the decreased levels of HTRA1 protein or functional HTRA1 protein are below the levels of HTRA1 protein or functional HTRA1 protein by no more than 1%, 5%, 10%, 20%, 25%, 30%, 40%, 50%, 60%, 70%, 80%, 90% or 100% of the levels expressed by the same cell type or tissue type in the reference control subject or population of reference control subjects.

In some embodiments, any of the treatment and/or prophylactic methods disclosed herein are applied to a subject. In some embodiments, the subject is a mammal. In some embodiments, the subject is a human. In some embodiments, the human is an adult. In some embodiments, the human is an elderly adult. In some embodiments, the human is at least 40, 45, 50, 55, 60, 65, 70, 75, 80, 85, 90, or 95 years of age. In particular embodiments, the human is at least 60 or 65 years of age.

In some embodiments, any of the treatment and/or prophylactic methods disclosed herein is for use in treatment of a patient having one or more mutations that causes macular degeneration (AMD) or that increases the likelihood that a patient develops AMD. In some embodiments, the one or more mutations are in the patient's HTRA1 gene.

In some embodiments, any of the treatment and/or prophylactic methods disclosed herein is for use in treatment of a subject having one or more mutations in the patient's HTRA1 gene. In some embodiments, the one or more mutations result in overexpression of the HTRA1 gene. As used herein, “mutations” encompasses polymorphisms that are associated with increased HTRA1 expression. In some embodiments, HTRA1 is expressed at a level at least 25%, 50%, 75%, 100%, 150%, 200%, 250%, 300%, 350%, 400%, 450%, or 500% greater in the subject having the disease or disorder as compared to the level in a control subject not having the disease or disorder. In some embodiments, the control subject is a subject of the same sex and/or of similar age as the subject having the disease or disorder. In some embodiments, the one or more mutations are not in the coding sequence for the HTRA1 gene. In some embodiments, the one or more mutations are in 10q26 in a human patient. In some embodiments, the one or more mutations correspond to any one or more of the following human polymorphisms: rs61871744; rs59616332; rs11200630; rs61871745; rs11200632; rs11200633; rs61871746; rs61871747; rs370974631; rs200227426; rs201396317; rs199637836; rs11200634; rs75431719; rs10490924; rs144224550; rs36212731; rs36212732; rs36212733; rs3750848; rs3750847; rs3750846; rs566108895; rs3793917; rs3763764; rs11200638; rs1049331; rs2293870; rs2284665; rs60401382; rs11200643; rs58077526; rs932275 and/or rs2142308.

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. As a result, the subject's vision is improved, or vision loss is arrested and/or ameliorated.

In a particular embodiment, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In another embodiment, a method of targeting one or more type(s) of ocular cells for gene augmentation therapy in a subject in need thereof is provided. In another embodiment, a method of targeting one or more type of ocular cells for gene suppression therapy in a subject in need thereof is provided. In yet another embodiment, a method of targeting one or more type of ocular cells for gene knockdown/augmentation therapy in a subject in need thereof is provided. In another embodiment, a method of targeting one or more type of ocular cells for gene correction therapy in a subject in need thereof is provided. In still another embodiment, a method of targeting one or more type of ocular cells for neurotropic factor gene therapy in a subject in need thereof is provided.

In any of the methods described herein, the targeted cell may be an ocular cell. In one embodiment, the targeted cell is a glial cell. In one embodiment, the targeted cell is an RPE cell. In another embodiment, the targeted cell is a photoreceptor. In another embodiment, the photoreceptor is a cone cell. In another embodiment, the targeted cell is a Muller cell. In another embodiment, the targeted cell is a bipolar cell. In yet another embodiment, the targeted cell is a horizontal cell. In another embodiment, the targeted cell is an amacrine cell. In still another embodiment, the targeted cell is a ganglion cell. In still another embodiment, the gene may be expressed and delivered to an intracellular organelle, such as a mitochondrion or a lysosome.

In some embodiments, any of the methods disclosed herein increase photoreceptor function. As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

In another embodiment, the method includes performing functional and imaging studies to determine the efficacy of the treatment. These studies include ERG and in vivo retinal imaging, as described in the examples below. In addition visual field studies, perimetry and microperimetry, pupillometry, mobility testing, visual acuity, contrast sensitivity, color vision testing may be performed.

In yet another embodiment, any of the above described methods is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, Sieving, P. A. et al, 2006. Proc. Natl. Acad. Sci. USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod derived cone viability factor 2. The secondary therapy can be administered before, concurrent with, or after administration of the ASO agents described above.

In some embodiments, any of the ASO agents or compositions disclosed herein is administered to a subject in combination with another therapeutic agent or therapeutic procedure. In some embodiments, the additional therapeutic agent is an anti-VEGF therapeutic agent (e.g., such as an anti-VEGF antibody or fragment thereof such as ranibizumab, bevacizumab or aflibercept), a vitamin or mineral (e.g., vitamin C, vitamin E, lutein, zeaxanthin, zinc or copper), omega-3 fatty acids, and/or Visudyne™. In some embodiments, the other therapeutic procedure is a diet having reduced omega-6 fatty acids, laser surgery, laser photocoagulation, submacular surgery, retinal translocation, and/or photodynamic therapy.

Kits

In some embodiments, any of the ASO agents disclosed herein is assembled into a pharmaceutical or diagnostic or research kit to facilitate their use in therapeutic, diagnostic or research applications. A kit may include one or more containers housing any of the ASO agents disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

### EXAMPLES

The disclosure now being generally described, it will be more readily understood by reference to the following examples, which are included merely for purposes of illustration of certain embodiments and embodiments of the present disclosure, and are not intended to limit the disclosure.

### Example 1

### Use of ASO Agents for Treating AMD

This study will evaluate the efficacy of an ASO agent comprising the nucleotide sequence of any one of SEQ ID NOs: 1-20 for treating patients with AMD. Patients with AMD will be treated with any of these ASO agents, or a control. The ASO agents will be administered at varying doses. The ASO agents will be administered by intravitreal injection in a solution of PBS with additional NaCl and pluronic. Patients will be monitored for improvements in AMD symptoms.

It is expected that the ASO treatments will improve the AMD symptoms.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

SEQUENCE LISTING

| | ASO sequence |
|---|---|
| SEQ ID NO: 1 | GTTATATTTATGGCGCAAAC |
| SEQ ID NO: 2 | CTGATTATGACGTCGTTTTC |
| SEQ ID NO: 3 | GCGAAACAATTCGATATGAA |
| SEQ ID NO: 4 | CTGTCACTTTCAAAGTGTTA |
| SEQ ID NO: 5 | GTCCGCGATAAAGTTATATT |
| SEQ ID NO: 6 | GGAATCACTGTGATCATGAT |
| SEQ ID NO: 7 | CTTATCAGATGGGATTGCAA |
| SEQ ID NO: 8 | TATATACGCTCCTGAGATCA |
| SEQ ID NO: 9 | GATTGCAAAGGAGATTCCAG |
| SEQ ID NO: 10 | TGTCCATTGATGCTGATTAT |
| SEQ ID NO: 11 | CTGTGTTTTGAAGGGAAAAC |
| SEQ ID NO: 12 | CTTTCCCTTTTAATGACGTC |
| SEQ ID NO: 13 | GTGGTCAATTTTGATGAGTG |
| SEQ ID NO: 14 | ATACTTCTTCTTGGTGATGG |
| SEQ ID NO: 15 | CATGATATCTTCATTACCCC |
| SEQ ID NO: 16 | AAACTGTTGGGATCTTCCTG |
| SEQ ID NO: 17 | GTCAATTTCTTCGGGAATCA |
| SEQ ID NO: 18 | TCTCGTTTAGAAAACGGAAG |
| SEQ ID NO: 19 | TGAGTGACATCATTCGGATA |
| SEQ ID NO: 20 | TCCGTGAGGAACTTTTTAAT |

Human HTRA1 Amino Acid Sequence-
GenBank Accession No. NP_002766.1
SEQ ID NO: 21
MQIPRAALLPLLLLLLAAPASAQLSRAGRSAPLAAGCP
DRCEPARCPPQPEHCEGGRARDACGCCEVCGAPEGAAC
GLQEGPCGEGLQCVVPFGVPASATVRRRAQAGLCVCAS
SEPVCGSDANTYANLCQLRAASRRSERLHRPPVIVLQR
GACGQGQEDPNSLRHKYNFIADVVEKIAPAVVHIELFR
KLPFSKREVPVASGSGFIVSEDGLIVTNAHVVTNKHRV
KVELKNGATYEAKIKDVDEKADIALIKIDHQGKLPVLL
LGRSSELRPGEFVVAIGSPFSLQNTVTTGIVSTTQRGG
KELGLRNSDMDYIQTDAIINYGNSGGPLVNLDGEVIGI
NTLKVTAGISFAIPSDKIKKFLTESHDRQAKGKAITKK
KYIGIRMMSLTSSKAKELKDRHRDFPDVISGAYIIEVI
PDTPAEAGGLKENDVIISINGQSVVSANDVSDVIKRES
TLNMVVRRGNEDIMITVIPEEIDP
Human HTRA1 Polynucleotide Sequence-
GenBank Accession No. NM_002775.4
SEQ ID NO: 22
CAATGGGCTGGGCCGCGCGGCCGCGCGCACTCGCACCC
GCTGCCCCCGAGGCCCTCCTGCACTCTCCCCGGCGCCG
CTCTCCGGCCCTCGCCCTGTCCGCCGCCACCGCCGCCG
CCGCCAGAGTCGCCATGCAGATCCCGCGCGCCGCTCTT
CTCCCGCTGCTGCTGCTGCTGCTGGCGGCGCCCGCCTC
GGCGCAGCTGTCCCGGGCCGGCCGCTCGGCGCCTTTGG
CCGCCGGGTGCCCAGACCGCTGCGAGCCGGCGCGCTGC
CCGCCGCAGCCGGAGCACTGCGAGGGCGGCCGGGCCCG
GGACGCGTGCGGCTGCTGCGAGGTGTGCGGCGCGCCCG
AGGGCGCCGCGTGCGGCCTGCAGGAGGGCCCGTGCGGC
GAGGGGCTGCAGTGCGTGGTGCCCTTCGGGGTGCCAGC
CTCGGCCACGGTGCGGCGGCGCGCGCAGGCCGGCCTCT
GTGTGTGCGCCAGCAGCGAGCCGGTGTGCGGCAGCGAC
GCCAACACCTACGCCAACCTGTGCCAGCTGCGCGCCGC
CAGCCGCCGCTCCGAGAGGCTGCACCGGCCGCCGGTCA
TCGTCCTGCAGCGCGGAGCCTGCGGCCAAGGGCAGGAA
GATCCCAACAGTTTGCGCCATAAATATAACTTTATCGC
GGACGTGGTGGAGAAGATCGCCCCTGCCGTGGTTCATA
TCGAATTGTTTCGCAAGCTTCCGTTTTCTAAACGAGAG
GTGCCGGTGGCTAGTGGGTCTGGGTTTATTGTGTCGGA
AGATGGACTGATCGTGACAAATGCCCACGTGGTGACCA
ACAAGCACCGGGTCAAAGTTGAGCTGAAGAACGGTGCC
ACTTACGAAGCCAAAATCAAGGATGTGGATGAGAAAGC
AGACATCGCACTCATCAAAATTGACCACCAGGGCAAGC
TGCCTGTCCTGCTGCTTGGCCGCTCCTCAGAGCTGCGG
CCGGGAGAGTTCGTGGTCGCCATCGGAAGCCCGTTTTC
CCTTCAAAACACAGTCACCACCGGGATCGTGAGCACCA
CCCAGCGAGGCGGCAAAGAGCTGGGGCTCCGCAACTCA
GACATGGACTACATCCAGACCGACGCCATCATCAACTA
TGGAAACTCGGGAGGCCCGTTAGTAAACCTGGACGGTG
AAGTGATTGGAATTAACACTTTGAAAGTGACAGCTGGA
ATCTCCTTTGCAATCCCATCTGATAAGATTAAAAAGTT
CCTCACGGAGTCCCATGACCGACAGGCCAAAGGAAAAG
CCATCACCAAGAAGAAGTATATTGGTATCCGAATGATG
TCACTCACGTCCAGCAAAGCCAAAGAGCTGAAGGACCG
GCACCGGGACTTCCCAGACGTGATCTCAGGAGCGTATA
TAATTGAAGTAATTCCTGATACCCCAGCAGAAGCTGGT
GGTCTCAAGGAAAACGACGTCATAATCAGCATCAATGG
ACAGTCCGTGGTCTCCGCCAATGATGTCAGCGACGTCA
TTAAAAGGGAAAGCACCCTGAACATGGTGGTCCGCAGG
GGTAATGAAGATATCATGATCACAGTGATTCCCGAAGA
AATTGACCCATAGGCAGAGGCATGAGCTGGACTTCATG
TTTCCCTCAAAGACTCTCCCGTGGATGACGGATGAGGA
CTCTGGGCTGCTGGAATAGGACACTCAAGACTTTTGAC
TGCCATTTTGTTTGTTCAGTGGAGACTCCCTGGCCAAC
AGAATCCTTCTTGATAGTTTGCAGGCAAAACAAATGTA
ATGTTGCAGATCCGCAGGCAGAAGCTCTGCCCTTCTGT
ATCCTATGTATGCAGTGTGCTTTTTCTTGCCAGCTTGG
GCCATTCTTGCTTAGACAGTCAGCATTTGTCTCCTCCT
TTAACTGAGTCATCATCTTAGTCCAACTAATGCAGTCG
ATACAATGCGTAGATAGAAGAAGCCCCACGGGAGCCAG
GATGGGACTGGTCGTGTTTGTGCTTTTCTCCAAGTCAG
CACCCAAAGGTCAATGCACAGAGACCCCGGGTGGGTGA
GCGCTGGCTTCTCAAACGGCCGAAGTTGCCTCTTTTAG
GAATCTCTTTGGAATTGGGAGCACGATGACTCTGAGTT
TGAGCTATTAAAGTACTTCTTACACATTGCAAAAAAAA
AAAAAAAAAA

1. An antisense oligomer (ASO) agent that targets an HTRA1 polynucleotide, wherein the HTRA1 polynucleotide encodes an HTRA1 polypeptide or functional fragment thereof, and wherein the ASO agent comprises a nucleotide sequence that is at least 80%, 85%, 90%, 93%, 95%, 97%, 98%, 99% or 100% identical to any one of SEQ ID NOs: 1-20.

2-4. (canceled)

5. The ASO agent of claim 1, wherein the ASO agent comprises at least 8, 9, 10, 11, 12, 13, 14, 15, 16, 17, 18 or 19 contiguous nucleotides from a nucleotide sequence of any one of SEQ ID NOs: 1-20.

6-10. (canceled)

11. The ASO agent of claim 1, wherein the ASO agent is capable of inhibiting the expression of HTRA1 protein by at least 5%, 10%, 15%, 25%, 30%, 35%, 40%, 45%, 50%, 55%, 60%, 65%, 70%, 75%, 80%, 85%, 90%, 95% or 100% as compared to the expression level of HTRA1 protein in the absence of the ASO agent.

12. The ASO agent of claim 1, wherein the ASO agent targets an HTRA1-encoding mRNA transcript or an HTRA1 pre-mRNA transcript.

13. (canceled)

14. The ASO agent of claim 1, wherein the ASO agent is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the ASO agent.

15-17. (canceled)

18. The ASO agent of claim 1, wherein the ASO agent is capable of reducing HTRA1 pre-mRNA transcript levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1 pre-mRNA transcript levels in the same cell type in the absence of the ASO agent.

19. The ASO agent of claim 1, wherein the ASO agent comprises one or more modified nucleotides.

20. (canceled)

21. The ASO agent of claim 1, wherein one or more nucleotides of the ASO agent are linked by modified internucleoside linkages or backbones.

22. (canceled)

23. The ASO agent of claim 1, wherein the ASO agent comprises 8 to 30 linked nucleosides and having a nucleobase sequence comprising a complementary region comprising at least 8 contiguous nucleobases complementary to a target region of equal length in an HTRA1 transcript.

24-27. (canceled)

28. The ASO agent of claim 1, wherein the ASO agent is capable of:
(i) ainterfering with polyadenylation of HTRA1 pre-mRNA;
(ii) inhibiting formation of the 5′-cap of HTRA1 pre-mRNA;
(iii) inhibiting splicing of HTRA1 pre-mRNA; and/or
(iv) activating RNase H-dependent degradation of a target HTRA mRNA transcript.

29-31. (canceled)

32. The ASO agent of claim 1, wherein the ASO agent is a gapmer or morpholino.

33. (canceled)

34. A vector comprising the ASO agent of claim 1.

35-39. (canceled)

40. A host cell comprising the vector of claim 1.

41. A method of treating a disease or disorder in a subject in need thereof, wherein the disease or disorder is associated with aberrantly expressed HTRA1, wherein the method comprises administering to the subject the ASO agent of any one of claims 1 33claim 1 or [[the]]a vector comprising the ASO agent of claim 1 or a vector comprising the ASO agent.

42. A method of treating a disease or disorder in a subject in need thereof, wherein HTRA1 is expressed at a level at least 5%, 10%, 25%, 50%, 75%, 100%, 150%, 200%, 250%, 300%, 350%, 400%, 450%, or 500% greater in the subject having the disease or disorder as compared to the level in a control subject not having the disease or disorder, wherein the method comprises administering to the subject the ASO agent of claim 1 or a vector comprising the ASO agent.

43. A method of treating age-related macular degeneration or polypoidal choroidal vasculopathy, wherein the method comprises administering to the subject the ASO agent of claim 1 or a vector comprising the ASO agent.

44. (canceled)

45. The method of claim 41, wherein the subject has one or more mutations in the HTRA1 gene.

46. The method of claim 45, wherein the one or more mutations are not in the coding sequence for the HTRA1 gene or wherein the one or more mutations are in 10q26 in a human subject.

47-60. (canceled)

61. A composition comprising a pharmaceutically acceptable carrier and (i) the ASO agent of claim 1 or (ii) a vector comprising the ASO agent.

62. The composition of claim 61, wherein the composition is substantially pyrogen free.


SEQUENCE LISTING (22 sequences)

SEQ ID NO: 1 - DNA, 20 bp, Homo sapiens
gttatatttatggcgcaaac

SEQ ID NO: 2 - DNA, 20 bp, Homo sapiens
ctgattatgacgtcgttttc

SEQ ID NO: 3 - DNA, 20 bp, Homo sapiens
gcgaaacaattcgatatgaa

SEQ ID NO: 4 - DNA, 20 bp, Homo sapiens
ctgtcactttcaaagtgtta

SEQ ID NO: 5 - DNA, 20 bp, Homo sapiens
gtccgcgataaagttatatt

SEQ ID NO: 6 - DNA, 20 bp, Homo sapiens
ggaatcactgtgatcatgat

SEQ ID NO: 7 - DNA, 20 bp, Homo sapiens
cttatcagatgggattgcaa

SEQ ID NO: 8 - DNA, 20 bp, Homo sapiens
tatatacgctcctgagatca

SEQ ID NO: 9 - DNA, 20 bp, Homo sapiens
gattgcaaaggagattccag

SEQ ID NO: 10 - DNA, 20 bp, Homo sapiens
tgtccattgatgctgattat

SEQ ID NO: 11 - DNA, 20 bp, Homo sapiens
ctgtgttttgaagggaaaac

SEQ ID NO: 12 - DNA, 20 bp, Homo sapiens
ctttcccttttaatgacgtc

SEQ ID NO: 13 - DNA, 20 bp, Homo sapiens
gtggtcaattttgatgagtg

SEQ ID NO: 14 - DNA, 20 bp, Homo sapiens
atacttcttcttggtgatgg

SEQ ID NO: 15 - DNA, 20 bp, Homo sapiens
catgatatcttcattacccc

SEQ ID NO: 16 - DNA, 20 bp, Homo sapiens
aaactgttgggatcttcctg

SEQ ID NO: 17 - DNA, 20 bp, Homo sapiens
gtcaatttcttcgggaatca

SEQ ID NO: 18 - DNA, 20 bp, Homo sapiens
tctcgtttagaaaacggaag

SEQ ID NO: 19 - DNA, 20 bp, Homo sapiens
tgagtgacatcattcggata

SEQ ID NO: 20 - DNA, 20 bp, Homo sapiens
tccgtgaggaactttttaat

SEQ ID NO: 21 - protein, 480 aa, Homo sapiens
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 22 - DNA, 2138 bp, Homo sapiens
caatgggctgggccgcgcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgcaaaaaaaaaaaaaaaaaa